(19) United States
(12) Patent Application Publication
(10) Pub. No.: US 2022/0151551 A1
(43) Pub. Date: May 19, 2022

(54) Systems and Methods for Memory Specificity with Virtual Reality

(71) Applicant: The Regents of the University of California, Oakland, CA (US)

(72) Inventors: Michelle G. Craske, Los Angeles, CA (US); Michael Treanor, Los Angeles, CA (US)

(73) Assignee: The Regents of the University of California, Oakland, CA (US)

(21) Appl. No.: 17/597,568

(22) PCT Filed: Jul. 10, 2020
(86) PCT No.: PCT/US2020/041645

§ 371 Date: Jan. 11, 2022

Related U.S. Application Data
(60) Provisional application No. 62/873,493, filed on Jul. 12, 2019.

(51) Int. Cl. A61B 5/00; G06F 3/01; A61B 5/024

(52) U.S. Cl. CPC A61B 5/4833; G06F 3/011; A61B 5/7475; A61B 5/486; A61B 5/02438

(57) ABSTRACT

Systems and methods for virtualized treatments in accordance with embodiments of the invention are illustrated. One embodiment includes a method for providing a virtualized treatment experience. The method includes steps for generating a treatment sequence includes a plurality of treatment elements, generating a virtualized environment for the generated treatment sequence, providing a plurality of treatment elements within the virtualized environment, and providing a set of one or more prompts for at least one treatment element of the plurality of treatment elements.

### CROSS-REFERENCE TO RELATED APPLICATIONS

The current application claims the benefit of and priority under 35 U.S.C. § 119(e) to U.S. Provisional Patent Application No. 62/873,493 entitled “Systems and Methods for Memory Specificity with Virtual Reality” filed Jul. 12, 2019. The disclosure of U.S. Provisional Patent Application No. 62/873,493 is hereby incorporated by reference in its entirety for all purposes.

### FIELD OF THE INVENTION

The present invention generally relates to treatments for neurological symptoms and, more specifically, treatments for neurological symptoms using virtual reality.

### BACKGROUND

Anhedonia is a transdiagnostic construct characterized by diminished capacity to experience joy or pleasure in individuals with depression, social anxiety, schizophrenia, and substance use disorder. Clinically-significant symptoms of anhedonia have been found to be a risk factor for suicide and poor treatment response in depressed individuals. Yet, existing treatments have had limited impact on anhedonia. Standard antidepressant medication treatments sometimes worsen anhedonic symptoms. The majority of evidence-based psychological therapies target heightened negative affect instead of deficits in positive affect. Although behavioral activation therapy is founded upon principles of positive reinforcement, evidence to date suggests that this treatment is relatively ineffective for positive affect.

The dysregulated reward circuitry underlying deficits in anhedonia presents itself as a prime target for treatment efforts. The NIH RDoC subconstructs operationalize these targets as reward anticipation (motivation), reward satiation (satisfaction) and reward learning, among others. Dopaminergic signaling plays a large role in motivation for reward and is primarily linked to the ventral tegmental area and nucleus accumbens. Reward learning is also associated with dopaminergic signaling and has been linked to such areas as the dorsolateral prefrontal cortex, anterior cingulate cortex, and orbitofrontal cortex.

There is compelling evidence for strong links between anhedonia and reward hyposensitivity. Specifically, anhedonic symptoms are associated with deficits in ventral striatum responsivity to anticipation of reward, and the behavioral effort expended to obtain rewards. Ventral striatum hypoactivity to positive stimuli is particularly related to anhedonic symptoms relative to depression symptoms more broadly and anhedonia is associated with reduced cardiac acceleration while viewing pleasant pictorial stimuli or imagining pleasant emotional scripts. For reward learning, blunted ventral striatal responses to instrumental conditioning tasks correlate with anhedonic symptoms as do impairments in a response bias to stimuli that are frequently rewarded.

### SUMMARY OF THE INVENTION

Systems and methods for virtualized treatments in accordance with embodiments of the invention are illustrated. One embodiment includes a method for providing a virtualized treatment experience. The method includes steps for generating a treatment sequence includes a plurality of treatment elements, generating a virtualized environment for the generated treatment sequence, providing a plurality of treatment elements within the virtualized environment, and providing a set of one or more prompts for at least one treatment element of the plurality of treatment elements.

In a further embodiment, the method further includes steps for receiving responses from a user and analyzing the received responses.

In still another embodiment, the method further includes steps for modifying the treatment sequence based on the analyzed received responses.

In a still further embodiment, the received responses are provided in responses to the set of prompts.

In yet another embodiment, receiving the responses includes monitoring reactions of a user using a set of one or more sensors during at least one treatment element.

In a yet further embodiment, the set of sensors includes at least one of a microphone, a video camera, and a heartrate sensor.

In another additional embodiment, the plurality of treatment elements includes at least one of a video and a rendered 3 dimensional (3D) virtual environment.

In a further additional embodiment, the virtualized environment includes a virtual reality (VR) environment.

In another embodiment again, the set of prompts includes a prompt to recall positive sensations felt during a treatment element.

In a further embodiment again, the set of prompts includes a prompt to recall positive sensations felt outside of the virtualized environment.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 conceptually illustrates a process for treatment sessions in accordance with an embodiment of the invention.

FIG. 2 illustrates an example of a virtualized treatment system that provides virtualized treatments in accordance with an embodiment of the invention.

FIG. 3 illustrates an example of a virtualized treatment element that provides virtualized treatments in accordance with an embodiment of the invention.

FIG. 4 illustrates an example of a virtualized treatment application that provides virtualized treatments in accordance with an embodiment of the invention.

### DETAILED DESCRIPTION

Turning now to the drawings, systems and methods in accordance with numerous embodiments of the invention can provide treatment for anhedonia through a virtualized environment (such as, but not limited to, in virtual reality (VR) or augmented reality (AR)). Although many of the examples described herein refer specifically to anhedonia, one skilled in the art will recognize that similar systems and methods can be used in a variety of applications, including (but not limited to) treatments for depression and other mental conditions, without departing from this invention.

Advances in neuroscience indicate specific targets that may underlie anhedonia that can be shifted through behavioral training. Systems and methods in accordance with a number of embodiments of the invention utilize behavioral training programs for anhedonia that can include exposure to positive or rewarding activities. In several embodiments, processes can provide memory recounting (or memory specificity training). An essential component is delivery of the exposure through virtualization technology, which allows repeated controlled immersion in experiences which are curated to enhance approach motivation towards and initial responsiveness to rewarding or positive experiences.

Neuroscience-based approaches have led to advances in the development of treatments targeting deficits in reward sensitivity. Treatments in accordance with some embodiments of the invention may target deficits in reward sensitivity such as processes described in “Treatment for anhedonia: a neuroscience driven approach” by Craske et al., published in the Journal of Consulting and Clinical Psychology, 87(5), 457-471, the disclosure from which relevant to positive affect treatment methods is hereby incorporated by reference in its entirety. Positive Affect Treatment (PAT) for patients with depression or anxiety has been shown to be more effective than cognitive behavioral therapy designed to reduce negative affect in terms of positive affect, as well as symptoms of depression, anxiety and stress. Behavioral activation, a core component of PAT, depends upon the availability of, and motivation to engage in, rewarding activities. The very nature of anhedonia lessens the availability and motivation to engage in the activities.

Virtualized treatments in accordance with a number of embodiments of the invention can include behavioral activation to rewarding activities combined with in-depth imaginal recounting to augment memory for savoring of reward. In a variety of embodiments, treatments can include cognitive training to search for positive features and imagine positive outcomes of everyday situations to strengthen anticipation or motivation for reward. Treatments in accordance with many embodiments of the invention can include compassion training to strengthen savoring of reward. Each treatment component can additionally target reward learning. Virtual reality offers a tool for guaranteed availability of rewarding activities that require less effort and therefore less motivation. Virtual reality can provide standardization and experimental control over therapeutic activities that are needed in order to evaluate the mechanisms of treatment.

### Methods for Treatment

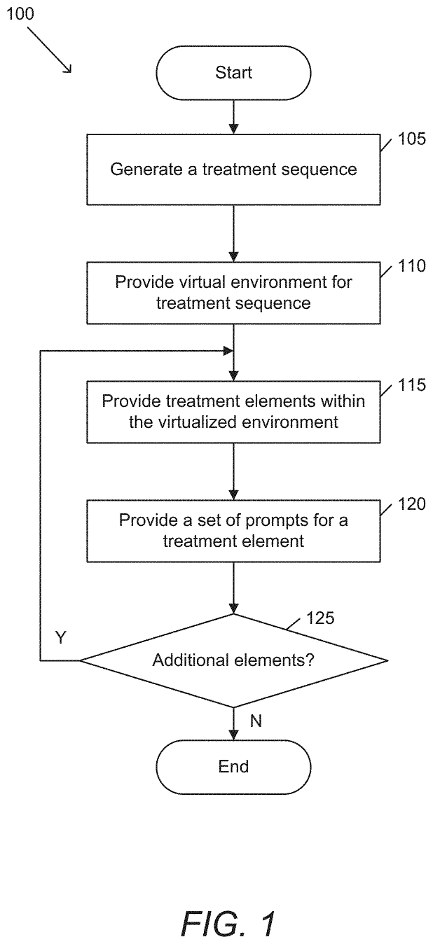

An example of a process for training (or treatment) sessions in accordance with several embodiments of the invention is illustrated in FIG. 1. In many embodiments, training sessions in accordance with many embodiments of the invention are to be performed across multiple sessions over a period of time (e.g., thirteen sessions over seven weeks).

Process 100 generates (105) a treatment sequence. Generating a treatment sequence in accordance with a number of embodiments of the invention can include analyzing patient data to generate a customized treatment sequence of treatment elements (such as, but not limited to, videos, a rendered 3D virtual environment, interactive sessions, questionnaires, music, vocal prompts, etc.) for a given patient. Treatment sequences in accordance with several embodiments of the invention can be customized based on a variety of factors including (but not limited to) the patient's medical history, previous treatment sessions, and/or medications. In certain embodiments, portions of a treatment sequence can be customized by an individual, either prior to beginning a session and/or during a given session. The generation of treatment sequences in accordance with a variety of embodiments of the invention can be based on various characteristics of the treatment elements (e.g., videos and/or music) nausea, valence, and/or arousal. In certain embodiments, the treatment sequences are adjusted between sessions, e.g., elements of earlier sessions may have higher positive valences that become increasingly neutral. In this way, participants can become trained in identifying the most positive features of the VR viewing even when surrounded by neutral stimuli.

Process 100 provides (110) a virtualized environment for the generated treatment sequence. Virtualized environments in accordance with some embodiments of the invention can include virtual reality environments and/or augmented reality environments. In several embodiments, virtualized environments can allow a user to feel as though they are in a different setting. Virtualized environments in accordance with many embodiments of the invention can also include non-visual markers, such as (but not limited to) soundscapes, smells, and/or tactile sensations.

Process 100 provides (115) treatment elements within the virtualized environment. In a number of embodiments, processes can monitor a user's reactions and/or state during each experience and analyze the reactions to determine prompts and/or new experiences.

Process 100 provides (120) a set of one or more prompts for a treatment element (or an experience). Prompts in accordance with a variety of embodiments of the invention can be used to encourage positive behaviors in a user such as (but not limited to) encouraging a user to think positively about the experience, to recall positive sensations during the experience, and/or to recall positive feelings. Prompts in accordance with some embodiments of the invention can be related to a provided experience and/or may prompt a user to think of a personal experience outside of the virtualized environment. In various embodiments, responses to prompts can be analyzed to determine an effectiveness of the treatment and/or to drive the selection of subsequent experiences (either within a same session or in a next session). In various embodiments, prompts can be provided within the virtualized environment. Alternatively, or conjunctively, prompts can be provided outside of the system in a variety of manners, such as (but not limited to) by another individual and/or through a separate application. Prompts in accordance with many embodiments of the invention can include related video, audio, and/or virtualized interactive presentations.

Process 100 determines (125) whether there are additional elements of the treatment sequence. In several embodiments, processes can determine that there are additional elements as long as a time limit has not been exceeded. When the process determines that there are additional elements of the treatment sequence, the process returns to step 115. When the process determines that there are no additional elements of the treatment sequence, the process ends.

While specific processes for virtualized treatment are described above, any of a variety of processes can be utilized to provide virtual treatments as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted. Although the above embodiments of the invention are described in reference to treatments for anhedonia, the techniques disclosed herein may be used in any type of treatment for neurological conditions, including depression, anxiety, and stress.

### Systems for Doing Something

### Virtualized Treatment System

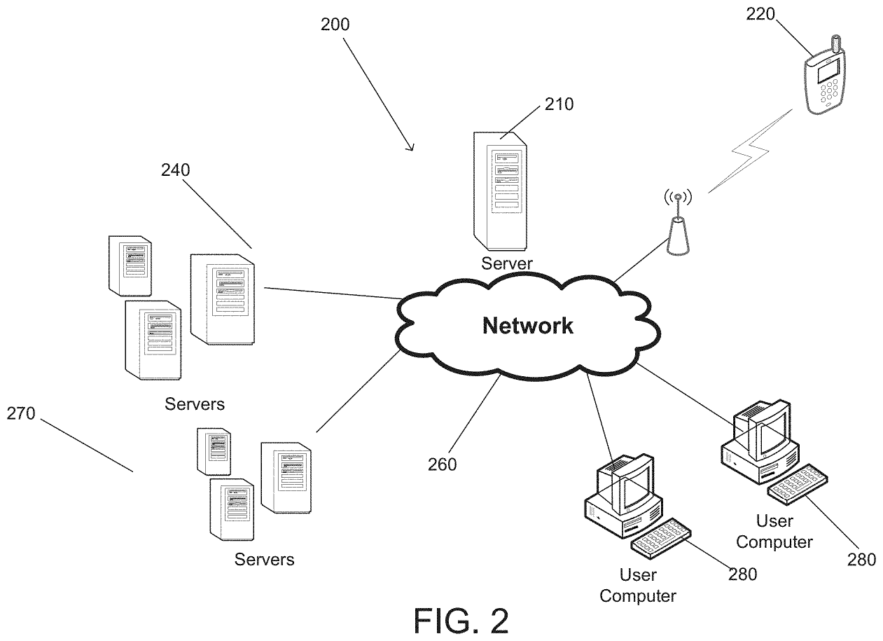

A virtualized treatment system that provides virtualized treatments in accordance with some embodiments of the invention is shown in FIG. 2. Network 200 includes a communications network 260. The communications network 260 is a network such as the Internet that allows devices connected to the network 260 to communicate with other connected devices. Server systems 210, 240, and 270 are connected to the network 260. Each of the server systems 210, 240, and 270 is a group of one or more servers communicatively connected to one another via internal networks that execute processes that provide cloud services to users over the network 260. For purposes of this discussion, cloud services are one or more applications that are executed by one or more server systems to provide data and/or executable applications to devices over a network. The server systems 210, 240, and 270 are shown each having three servers in the internal network. However, the server systems 210, 240 and 270 may include any number of servers and any additional number of server systems may be connected to the network 260 to provide cloud services. In accordance with various embodiments of this invention, a virtualized treatment system that uses systems and methods that provide virtualized treatments in accordance with an embodiment of the invention may be provided by a process being executed on a single server system and/or a group of server systems communicating over network 260. Treatment sequences in accordance with several embodiments of the invention can be generated at a server and provided to a patient at a client device. In various embodiments, responses from individuals can be collected at client devices and then gathered and analyzed at a cloud service.

Users may use personal devices 280 and 220 that connect to the network 260 to perform processes that provide virtualized treatments in accordance with various embodiments of the invention. In the shown embodiment, the personal devices 280 are shown as desktop computers that are connected via a conventional “wired” connection to the network 260. However, the personal device 280 may be a desktop computer, a laptop computer, a smart television, an entertainment gaming console, or any other device that connects to the network 260 via a “wired” connection. The mobile device 220 connects to network 260 using a wireless connection. A wireless connection is a connection that uses Radio Frequency (RF) signals, Infrared signals, or any other form of wireless signaling to connect to the network 260. In FIG. 2, the mobile device 220 is a mobile telephone. However, mobile device 220 may be a mobile phone, Personal Digital Assistant (PDA), a tablet, a smartphone, or any other type of device that connects to network 260 via wireless connection without departing from this invention.

While specific implementations of a virtualized treatment system have been described above with respect to 2, there are numerous configurations of such systems, including, but not limited to, those where the entire process is performed on a single device, and/or any other configuration as appropriate to the requirements of a given application. As can readily be appreciated the specific computing system used to provide virtualized treatments is largely dependent upon the requirements of a given application and should not be considered as limited to any specific computing system(s) implementation.

### Virtualized Treatment Element

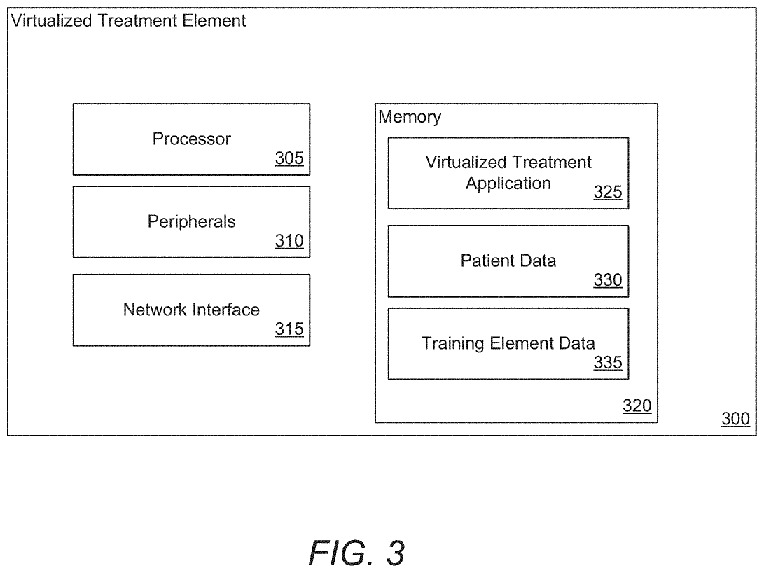

An example of a virtualized treatment element that executes instructions to perform processes that provide interaction with other devices connected to a network and/or for providing training tasks in accordance with various embodiments of the invention is shown in FIG. 3. Virtualized treatment elements in accordance with many embodiments of the invention can include (but are not limited to) one or more of mobile devices, servers, virtual reality (VR) systems, and computers. Virtualized treatment element 300 includes processor 305, peripherals 310, network interface 315, and memory 320.

One skilled in the art will recognize that a particular virtualized treatment element may include other components that are omitted for brevity without departing from this invention. The processor 305 can include (but is not limited to) a processor, microprocessor, controller, or a combination of processors, microprocessor, and/or controllers that performs instructions stored in the memory 320 to manipulate data stored in the memory. Processor instructions can configure the processor 305 to perform processes in accordance with certain embodiments of the invention.

Peripherals 310 can include any of a variety of components for capturing data, such as (but not limited to) cameras, displays, and/or sensors. In a variety of embodiments, peripherals can be used to gather inputs and/or provide outputs. Network interface 315 allows virtualized treatment element 300 to transmit and receive data over a network based upon the instructions performed by processor 305. Peripherals and/or network interfaces in accordance with many embodiments of the invention can be used to gather inputs that can be used for a variety of purposes including (but not limited to) to capture and/or analyze an individual's responses to a virtualized treatment sequence and/or to prompts provided as a part of the treatment sequence.

Memory 320 includes a virtualized treatment application 325, patient data 330, and training element data 335. Virtualized treatment applications in accordance with several embodiments of the invention can be used to generate and/or provide virtualized treatments. In several embodiments, patient data can include various information regarding an individual including (but not limited to) medical history, responses to previous treatment, and/or personal preferences. Training element data in accordance with several embodiments of the invention can include (but is not limited to) video, audio, text, prompts, 3D models, and/or other elements that can be provided as a part of a virtualized treatment sequence.

Although a specific example of a virtualized treatment element 300 is illustrated in FIG. 3, any of a variety of virtualized treatment elements can be utilized to perform processes for providing virtualized treatments similar to those described herein as appropriate to the requirements of specific applications in accordance with embodiments of the invention.

### Virtualized Treatment Application

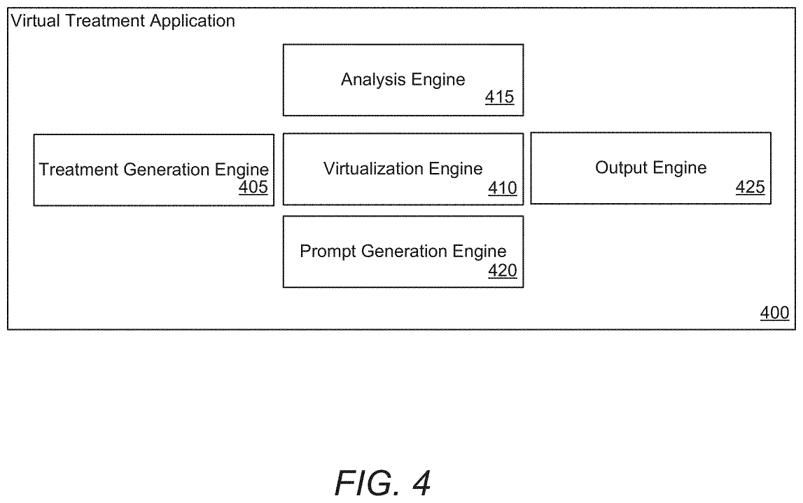

An example of a virtualized treatment application for providing virtual treatment experiences in accordance with an embodiment of the invention is illustrated in FIG. 4. Virtualized treatment application 400 includes treatment generation engine 405, virtualization engine 410, analysis engine 415, prompt generation engine 420, and output engine 425.

Treatment generation engines in accordance with numerous embodiments of the invention can be used to generate a treatment sequence for a session with an individual. Treatment sequences can be tailored to an individual based on numerous factors including (but not limited to) medical history, motion sensitivity, levels of depression, and/or progress within a treatment progression of treatment sessions. In several embodiments, generating treatment sequences can include (but is not limited to) selecting media content and/or generating prompts (e.g., via a prompt generation engine).

In some embodiments, virtualization engines can be used to generate virtualized environments (e.g., in virtual and/or augmented reality) for a treatment session. Virtualized environments in accordance with some embodiments of the invention can be used to allow a user to feel as though they are in a different, potentially challenging, environment from the safety and comfort of their own home. Virtualized environments in accordance with many embodiments of the invention can include various elements including, but not limited to, visual, auditory, olfactory, and tactile elements.

Analysis engines in accordance with various embodiments of the invention can be used to analyze a user's responses to a treatment session. Responses in accordance with numerous embodiments of the invention can include prompted responses (e.g., to specific prompts) provided within a session, as well as other responses (e.g., changes in heart rate, signs of stress, etc.) that are detected from a user (e.g., via video analysis, audio analysis, other sensory inputs). Analyses can be used in a variety of manners, such as (but not limited to) to direct prompts provided to the user, to modify a treatment sequence, and/or to be provided to a professional for further analysis.

In a variety of embodiments, prompt generation engines can be used to provide prompts to a user during a treatment session. Prompts in accordance with numerous embodiments of the invention can be provided within a virtualized environment (e.g., as text, as instructions from an avatar, etc.). In certain embodiments, prompts can direct a user to recall positive emotions felt during a treatment element and/or to recall positive emotions felt during an experience outside of the virtualized environment.

Output engines in accordance with several embodiments of the invention can provide a variety of outputs to a user, including (but not limited to) media outputs as a part of a treatment session and/or prompts for a user. In a variety of embodiments, output engines can provide analyses of a user's response to a professional for review, either at the same device or to another device (e.g., via a network).

Although specific methods of virtualized treatment are discussed above, many different methods of virtualized treatment can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for providing a treatment experience, the method comprising:
generating a treatment sequence comprising a plurality of treatment elements;
generating a virtualized environment for the generated treatment sequence;
providing a plurality of treatment elements within the virtualized environment; and
providing a set of one or more prompts for at least one treatment element of the plurality of treatment elements.

2. The method of claim 1 further comprising:
receiving responses from a user; and
analyzing the received responses.

3. The method of claim 2 further comprising modifying the treatment sequence based on the analyzed received responses.

4. The method of claim 2, wherein the received responses are provided in responses to the set of prompts.

5. The method of claim 2, wherein receiving the responses comprises monitoring reactions of a user using a set of one or more sensors during at least one treatment element.

6. The method of claim 5, wherein the set of sensors comprises at least one of a microphone, a video camera, and a heartrate sensor.

7. The method of claim 1, wherein the plurality of treatment elements comprises at least one of a video and a rendered 3 dimensional (3D) virtual environment.

8. The method of claim 1, wherein the virtualized environment comprises a virtual reality (VR) environment.

9. The method of claim 1, wherein the set of prompts comprises a prompt to recall positive sensations felt during a treatment element.

10. The method of claim 1, wherein the set of prompts comprises a prompt to recall positive sensations felt outside of the virtualized environment.

11. A non-transitory machine readable medium containing processor instructions for providing a treatment experience, where execution of the instructions by a processor causes the processor to perform a process that comprises:
generating a treatment sequence comprising a plurality of treatment elements;
generating a virtualized environment for the generated treatment sequence;
providing a plurality of treatment elements within the virtualized environment; and
providing a set of one or more prompts for at least one treatment element of the plurality of treatment elements.

12. The non-transitory machine readable medium of claim 11, wherein the process further comprises:
receiving responses from a user; and
analyzing the received responses.

13. The non-transitory machine readable medium of claim 12, wherein the process further comprises modifying the treatment sequence based on the analyzed received responses.

14. The non-transitory machine readable medium of claim 12, wherein the received responses are provided in responses to the set of prompts.

15. The non-transitory machine readable medium of claim 12, wherein receiving the responses comprises monitoring reactions of a user using a set of one or more sensors during at least one treatment element.

16. The non-transitory machine readable medium of claim 15, wherein the set of sensors comprises at least one of a microphone, a video camera, and a heartrate sensor.

17. The non-transitory machine readable medium of claim 11, wherein the plurality of treatment elements comprises at least one of a video and a rendered 3 dimensional (3D) virtual environment.

18. The non-transitory machine readable medium of claim 11, wherein the virtualized environment comprises a virtual reality (VR) environment.

19. The non-transitory machine readable medium of claim 11, wherein the set of prompts comprises a prompt to recall positive sensations felt during a treatment element.

20. The non-transitory machine readable medium of claim 11, wherein the set of prompts comprises a prompt to recall positive sensations felt outside of the virtualized environment.
